(19) United States
(12) Patent Application Publication
(10) Pub. No.: US 2021/0219915 A1
(43) Pub. Date: Jul. 22, 2021

(54) BIOLOGICAL INFORMATION MEASUREMENT DEVICE, BIOLOGICAL INFORMATION MEASUREMENT SYSTEM, AND INSERTER

(71) Applicant: PHC Holdings Corporation, Tokyo (JP)

(72) Inventors: Masahiro KOUGE, Ehime (JP); Seiichiro IKETANI, Ehime (JP); Akira NISHIO, Ehime (JP); Makoto OKAZAKI, Ehime (JP); Ryuji SHIMIZU, Ehime (JP)

(21) Appl. No.: 17/271,385

(22) PCT Filed: Aug. 23, 2019
(86) PCT No.: PCT/JP2019/033024

§ 371 Date: Feb. 25, 2021

(30) Foreign Application Priority Data
Sep. 5, 2018 (JP) 2018-166173
Sep. 5, 2018 (JP) 2018-166175

(51) Int. Cl. A61B 5/00; A61B 17/34

(52) U.S. Cl. CPC A61B 5/6833; A61B 17/3403; A61B 5/14532; A61B 2562/164; A61B 2562/242; A61B 5/6824

(57) ABSTRACT

The biological information measurement device comprises: an adhesive tape having on its bottom side an adhesive surface to be affixed to the skin of a user; a mounting substrate disposed on the top side of the adhesive tape; a guide needle insertion cylinder provided on the top side of the adhesive tape, on which the mounting substrate is held; a sensor electrically connected to the mounting substrate for measuring biological information; and a cover covering the top side of the mounting substrate. The bottom side of the mounting substrate is in a state of not being affixed to the top side of the adhesive tape, and the adhesive tape and the cover are more stretchable than the mounting substrate.

### TECHNICAL FIELD

The present invention relates to a biological information measurement device, a biological information measurement system used for continuous blood glucose measurement, for example, and an inserter.

### BACKGROUND ART

A conventional biological information measurement device comprised an adhesive tape having on its bottom side an adhesive surface that was affixed to the skin of user, a mounting substrate that was on the top side of the adhesive tape, and a sensor that was electrically connected to the mounting substrate and a part of which was transferred to a side of the skin of user (see, for example, Patent Literature 1 below).

Also, a conventional biological information measurement system comprised an adhesive tape, a mounting substrate disposed on the top side of the adhesive tape, a sensor electrically connected to the mounting substrate, and a cover that covered the top side of the mounting substrate. The configuration was such that the biological information measurement device was housed within an inserter, and the biological information measurement device was mounted on the human body by operating this inserter (see, for example, Patent Literature 1 below).

### CITATION LIST

### Patent Literature

Patent Literature 1: US Patent Application Publication No. 2017/0027514, specification

Patent Literature 2: Japanese Unexamined Patent Application Publication No. 2015-509011

### SUMMARY

### Technical Problem

In Patent Literature 1, a flexible substrate is used as a mounting substrate. Therefore, the flexible substrate bends to conform to the shape of the body part to which the biological information measurement device is attached, and this is anticipated to make the attachment to the skin more comfortable. After this, the flexible substrate and adhesive tape are covered with the cover.

However, with the above-mentioned conventional configuration, improved comfort cannot be expected during use beyond the initial attachment. The reason for this is that when the mounting substrate is a flexible substrate, the flexible substrate bends to conform to the shape of the attachment portion when attached to the user's body. Accordingly, at the time of attachment, the biological information measurement device is worn in a state of being fitted to the body, but the skin, bones, and muscles of the body move around considerably in the course of daily activities, so the flexible substrate was bent into one shape at the time of attachment may inhibit the displacement of the underlying skin. For instance, the flexible substrate inhibit displacement of the skin when the skin tries to stretch, and this can end up causing discomfort to the user, so improved comfort is desirable.

### Solution to Problem

In view of this, a first object of the first invention is to improve the comfort of wearing a biological information measurement device.

Meanwhile, in Patent Literature 2, the configuration is such that a biological information measurement device is housed within an inserter and attached to the user's body. Accordingly, the inserter ends up being larger, and as a result a problem is that the device is not as convenient to use.

In view of this, a second object of the second invention is to improve convenience when a biological information measurement device is attached.

In order to achieve the first object, the first invention comprises an adhesive tape having on its bottom side an adhesive surface to be affixed to the skin of user, a mounting substrate that is disposed on the top side of the adhesive tape, a sensor that is electrically connected to the mounting substrate and measures biological information, and a cover that covers the top side of the mounting substrate. The bottom side of the mounting substrate in this state is not bonded to the top side of the adhesive tape, and the adhesive tape and the cover are more stretchable than the mounting substrate.

With this configuration, the first object is achieved.

In order to achieve the second object, the second invention is a biological information measurement system comprising a biological information measurement device that measures biological information about a user, and an inserter that is placed on the upper surface of the biological information measurement device and is used to affix the biological information measurement device to the skin of the user, wherein the biological information measurement device has an adhesive tape having on its bottom side an adhesive surface to be affixed to the skin of user, a mounting substrate that is disposed on the top side of the adhesive tape, a substrate holding portion that is provided on the top side of the adhesive tape and that holds the mounting substrate, a sensor that is electrically connected to the mounting substrate and is used to measure biological information, and a cover that covers the top side of the mounting substrate and has a through-hole formed in the substrate holding portion. The inserter has a base body that is mounted on the upper surface of the cover of the biological information measurement device at a position including the through-hole, and that is supported by the upper end portion of the substrate holding portion of the biological information measurement device, and a flexible top body that is mounted on the base body and is elastically deformed when pressed during the mounting of the biological information measurement device on the skin.

With this configuration, the second object is achieved.

### Advantageous Effects

As described above, in the first invention, the adhesive tape affixed to the skin of user and the cover on the top side are stretchable, and the mounting substrate located between the adhesive tape and the cover is held by the substrate holding portion provided on the top side of the adhesive tape. Furthermore, since the bottom side of the mounting substrate is not bonded to the top side of the adhesive tape, the biological information measurement device is more comfortable when worn on the skin.

That is, since a very stretchable adhesive tape and cover are used, when the biological information measurement device is attached to the skin, it can be attached in a state of conforming to the skin. Also, if the body should be displaced (move) after attachment, stretching of the adhesive tape and the cover will minimize discomfort to the wearer, so the biological information measurement device can be worn more comfortably than a conventional device.

Also, as described above, in the second invention, the inserter comprises a base body that is mounted on the upper surface of the biological information measurement device, and a flexible top body that is mounted on the base body.

That is, since the inserter is mounted on the upper surface of the biological information measurement device, the inserter can smaller in size, and the biological information measurement device can be easily mounted with a finger by using this inserter.

As a result, the biological information measurement device is much more convenient during attachment than a conventional device.

### BRIEF DESCRIPTION OF DRAWINGS

FIG. 1 is an oblique view of the packaged state of the biological information measurement device according to Embodiment 1 of the present invention;

FIG. 2 is an oblique view of the biological information measurement device in FIG. 1;

FIG. 3 is a cross section of the biological information measurement device in FIG. 1;

FIG. 4 is a cross section of the biological information measurement device in FIG. 1;

FIG. 5 is a cross section of the biological information measurement device in FIG. 1;

FIG. 6 is a cross section of the biological information measurement device in FIG. 1;

FIG. 7 is an exploded oblique view of the biological information measurement device in FIG. 1;

FIG. 8 is an exploded oblique view of the biological information measurement device in FIG. 1;

FIG. 9 is an oblique view of the usage state of the biological information measurement device in FIG. 1;

FIG. 10 is an oblique view of the usage state of the biological information measurement device in FIG. 1;

FIG. 11 is an oblique view of the usage state of the biological information measurement device in FIG. 1;

FIG. 12 is an oblique view of the usage state of the biological information measurement device in FIG. 1;

FIG. 13 is a cross section of the usage state of the biological information measurement device in FIG. 1;

FIG. 14 is an exploded oblique view of the biological information measurement device in FIG. 1;

FIG. 15 is a detail cross section of the biological information measurement device in FIG. 1;

FIG. 16 is a cross section of the usage state of the biological information measurement device in FIG. 1;

FIG. 17 is a cross section of the usage state of the biological information measurement device in FIG. 1;

FIG. 18 is an oblique view of the usage state of the biological information measurement device in FIG. 1;

FIG. 19 is an oblique view of the usage state of the biological information measurement device in FIG. 1;

FIG. 20 is a cross section of the usage state of the biological information measurement device in FIG. 1;

FIG. 21 is an oblique view of the usage state of the biological information measurement device in FIG. 1;

FIG. 22 is a cross section of the biological information measurement device in

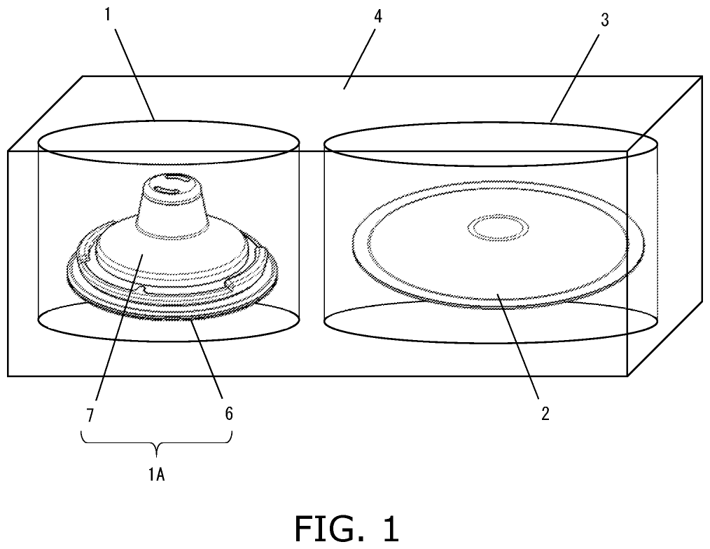

FIG. 1;

FIG. 23 is an oblique view of an overtape of the biological information measurement device in FIG. 1;

FIG. 24 is a cross section of the biological information measurement device in FIG. 1 in a state where an overtape has been attached;

FIG. 25 is an oblique view of the state when the biological information measurement device in FIG. 1 is attached; and

FIG. 26 is an electrical circuit diagram of the biological information measurement device in FIG. 1.

### DESCRIPTION OF EMBODIMENTS

Embodiments of the present invention will now be described in detail with reference to the drawings.

### Embodiment 1

A biological information measurement system 1A including a biological information measurement device 6 and an inserter 7 of this embodiment will now be described with reference to the drawings.

In the following description, terms indicating a specific direction or position are used (such as “top,” “bottom,” “right,” “left,” as well as other terms including those terms), but the use of these terms is intended to facilitate an understanding of the disclosure with reference to the drawings, and the meaning of these terms is not intended to limit the technical scope of the invention.

### (1) Configuration of Biological Information Measurement System 1A

The biological information measurement system 1A of this embodiment is used, for example, to attach the biological information measurement device 6 that measures blood glucose level and other such biological information to a user's upper arm or the like by using the inserter 7.

FIG. 1 shows a state in which a sterilized packaging 1 containing the biological information measurement device 6 and the inserter 7, and a sterilized packaging 3 containing an overtape 2 are housed in a container 4. That is, the biological information measurement device is transported while kept in a sterile state in the container 4 as shown in FIG. 1 before being attached to the skin 5 of the upper arm of the user whose biological information is to be measured, as shown in FIG. 25. In this embodiment, the biological information measurement system 1A is constituted by the biological information measurement device 6 and the inserter 7.

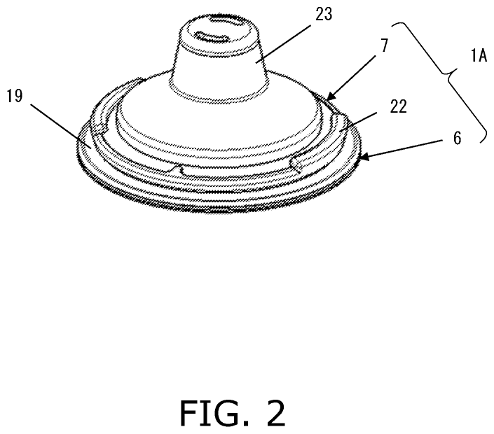

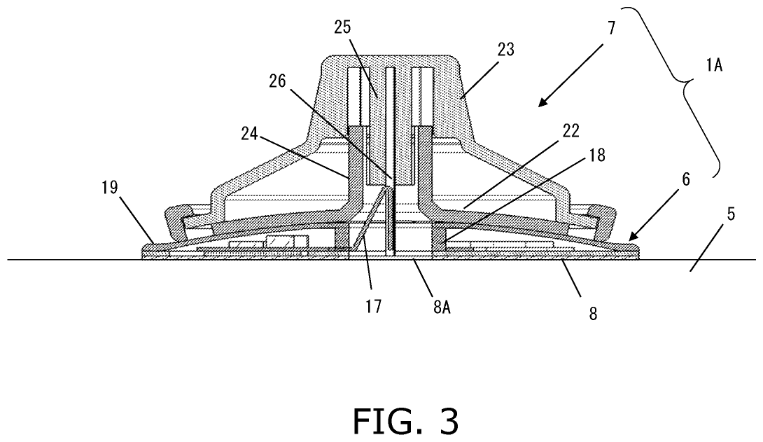

FIGS. 2 and 3 show the biological information measurement device 6 and the inserter 7 constituting the biological information measurement system 1A.

As shown in FIGS. 2 and 3, the inserter 7 used for attaching the biological information measurement device 6 to the upper arm, etc., of the user is detachably mounted on the upper surface of the biological information measurement device 6. Then, as mentioned above, the biological information measurement device 6 and the inserter 7 are held in the sterilized packaging 1 before use, as shown in FIG. 1.

### (2-1) Configuration of Biological Information Measurement Device 6

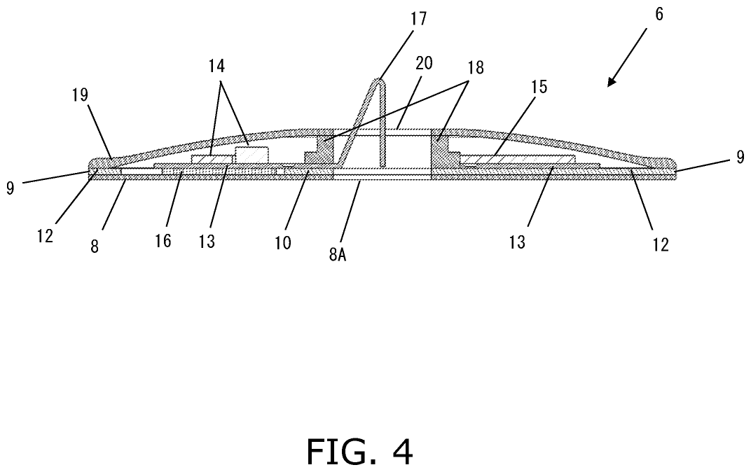

The biological information measurement device 6 has the configuration shown in FIGS. 4 to 8.

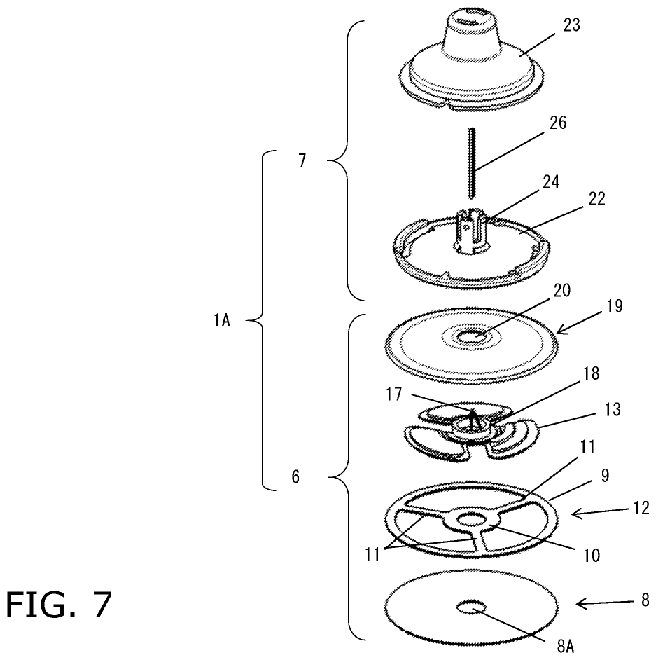

That is, as shown in FIGS. 4 and 7, the biological information measurement device 6 comprises an adhesive tape 8, a base 12, a mounting substrate 13, a sensor 17, a guide needle insertion cylinder 18 (an example of a substrate holding portion), and a cover 19.

FIG. 26 is an electrical block diagram of the biological information measurement device 6.

The measured value obtained by a measuring unit 30 using the sensor 17 is stored in a memory unit 32 via a control unit 31. Also, the measured value stored in the memory unit 32 is transmitted through a communication unit 33 to a mobile device 34.

As shown in FIG. 7, the substantially circular adhesive tape 8 is provided on the skin 5 side of the user's body in the biological information measurement device 6. The adhesive tape 8 is formed from a stretchable material such as rubber, an elastomer, a non-woven fabric, or a pressure-sensitive adhesive material. A through-hole 8A is provided in the center of the adhesive tape 8.

A base 12 in which the outer peripheral circle portion 9 and the inner peripheral circle portion 10 are connected by bridge portions 11 at intervals of 120 degrees as shown in FIG. 7 is provided on the adhesive tape 8.

The outer peripheral circle portion 9 forms the outermost peripheral portion of the substantially annular base 12, and is disposed concentrically with the inner peripheral circle portion 10. Also, the outer peripheral circle portion 9 and the inner peripheral circle portion 10 are connected to each other by three bridge portions 11 disposed in the radial direction.

The lower surface of the adhesive tape 8 is an entirely adhesive surface. On the other hand, the upper surface of the adhesive tape 8 is provided with an adhesive surface only on the outer peripheral circle portion 9, the inner peripheral circle portion 10, and the bridge portions 11. Consequently, the base 12 is held in a state of being bonded to the adhesive tape 8.

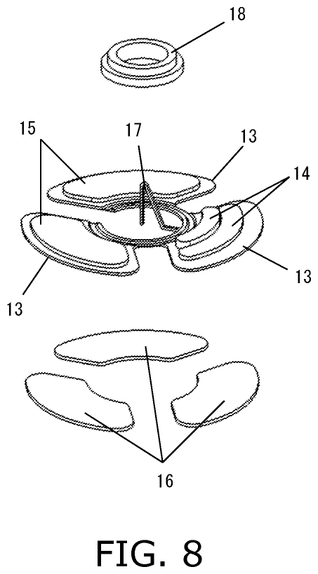

Next, as shown in FIGS. 4 and 7, the mounting substrate 13 is bonded onto the inner peripheral circle portion 10 of the base 12. As shown in FIG. 8, the mounting substrate 13 is divided into three parts at intervals of substantially equal angles (120 degrees). Electronic components 14 and a battery 15 are disposed on the upper surfaces of the mounting substrate 13 divided into three parts. As shown in FIG. 7, the mounting substrate 13 divided into three parts is disposed between the bridge portions 11 disposed at intervals of 120 degrees on the base 12.

More precisely, the three parts of the mounting substrate 13 are substantially fan shaped, each extending over an angular range of slightly less than 120 degrees. Therefore, between the bridge portions 11, the three parts of the mounting substrate 13 are disposed in a state of non-contact with the bridge portions 11, so as not to overlap the bridge portions 11 in top view.

Consequently, the spacer 16 disposed on the back side of the mounting substrate 13 can be disposed so as not to interfere with and ride up onto the bridge portions 11.

The mounting substrate 13 is formed from a resin such as polyimide or polyester used for flexible substrates. These materials are flexible in the bending direction, but they are not stretchable in a direction substantially parallel to the substrate surface after being attached to the skin 5.

Also, as shown in FIG. 8, fan-shaped spacers 16 are bonded to the bottom side of the three parts of the mounting substrate 13, that is, to the surface on the adhesive tape 8 side. Specifically, the mounting substrate 13 is in a state of being disposed on the adhesive tape 8 in an unbonded state via the spacers 16. That is, when the mounting substrate 13 is bonded, the adhesive tape 8 is less likely to be affected by the mounting substrate 13, which has no stretchability in the direction along the surface of the skin 5, which would hinder the stretching of the adhesive tape 8.

Also, the base 12 is formed from a stretchable material such as rubber or an elastomer. The base 12 is formed by connecting the outer peripheral circle portion 9 and the inner peripheral circle portion 10 to each other via the bridge portions 11 at intervals of 120 degrees as shown in FIG. 7. Accordingly, even when the base 12 is bonded to the upper surface of the adhesive tape 8, the stretching of the adhesive tape 8 will not be hindered by the stretchable base 12.

As shown in FIG. 4, the sensor 17 is electrically connected to the mounting substrate 13. The central portion of the sensor 17 is bent upward, and the distal end of the leg portion of the sensor 17 is held in a state of being inserted into the through-hole 8A in the adhesive tape 8.

The guide needle insertion cylinder 18 (an example of a substrate holding portion) is adhesively fixed to the top side of the mounting substrate 13 corresponding to the inner peripheral circle portion 10 of the base 12. That is, the mounting substrate 13 is held on the outer periphery of the guide needle insertion cylinder 18. The guide needle insertion cylinder 18 has a cylindrical shape. The central axis of the guide needle insertion cylinder 18 is disposed in a state of coinciding with the central axis of the through-hole 8A in the adhesive tape 8. As shown in FIG. 4, the central portion of the sensor 17 is disposed inside the guide needle insertion cylinder 18.

The guide needle insertion cylinder 18 is from a resin or the like, for example. That is, the guide needle insertion cylinder 18 is preferably formed from a material that is harder than the adhesive tape 8, the base 12, the mounting substrate 13, and the cover 19.

Consequently, since the guide needle holding portion 25, which holds a guide needle 26, is formed from a hard material that is not readily deformed, the guide needle 26 will not move around when inserted into the skin, and this allows the guide needle 26 to be inserted into the skin 5 (discussed below) stably and accurately.

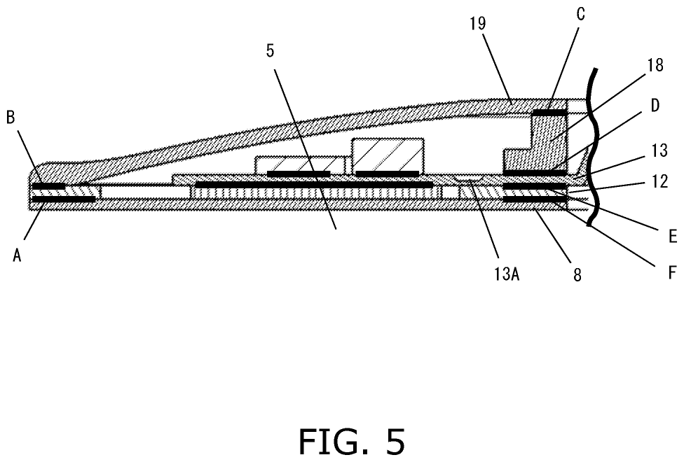

As shown in FIG. 5, the upper surface of the adhesive tape 8 and the lower surface of the base 12 are bonded together at a joint A of the outer peripheral edge portion. As shown in FIG. 5, the upper surface of the base 12 and the lower surface of the cover 19 are bonded together at a joint B of the outer peripheral edge portion. That is, the adhesive tape 8 and the cover 19 are bonded together via the base 12 at the outer peripheral edge portion.

As shown in FIG. 5, the lower surface of the cover 19 and the substantially cylindrical upper surface of the guide needle insertion cylinder 18 provided near the center of the substantially disk-shaped biological information measurement device 6 are bonded together at a position on the inner peripheral side (joint C).

The lower surface of the guide needle insertion cylinder 18 and the upper surface of the mounting substrate 13 are bonded together at a position on the inner peripheral side (joint D), similar to the joint C.

The lower surface of the mounting substrate 13 and the upper surface of the base 12 are bonded together at a position below the joint D of the guide needle insertion cylinder 18 (joint E).

The upper surface of the adhesive tape 8 and the lower surface of the base 12 are bonded together at a position below the joint D of the guide needle insertion cylinder 18 (joint F).

That is, the adhesive tape 8, the cover 19, and the base 12 are bonded together at the outer peripheral edge portion and near the center position of the substantially disk-shaped biological information measurement device 6.

The mounting substrate 13 is bonded to the adhesive tape 8, the cover 19, and the base 12 via the guide needle insertion cylinder 18 in the vicinity of the central portion of the substantially disk-shaped biological information measurement device 6.

Furthermore, the cover 19 is disposed on the mounting substrate 13 as shown in FIG. 7. The cover 19 is formed from a stretchable material such as rubber or an elastomer. As shown in FIG. 7, a through-hole 20 having substantially the same inside diameter as the through-hole 8A is provided in the central portion of the cover 19.

The outer peripheral edge of the through-hole 20 on the bottom side is adhesively fixed to the upper end surface of the substantially cylindrical guide needle insertion cylinder 18. Also, the lower surface of the cover 19 on the outer peripheral side is adhesively fixed to the upper surface of the outer peripheral circle portion 9 of the base 12. The cover 19 is disposed in state of not being bonded to the mounting substrate 13. That is, the cover 19 is disposed in a state of being free to stretch, without being affected by the mounting substrate 13.

In this embodiment, the mounting substrate 13 is disposed on the adhesive tape 8 via the spacers 16 bonded to the bottom side. Therefore, the mounting substrate 13 is put in place without being bonded to the adhesive tape 8. Also, on the upper surface of the adhesive tape 8, an adhesive surface is provided only on the outer peripheral circle portion 9, the inner peripheral circle portion 10, and the bridge portions 11. Accordingly, the mounting substrate 13 and the spacers 16 are not bonded to the adhesive tape 8. Also, the mounting substrate 13 is put in place in a state of not being bonded to the lower surface of the cover 19.

Consequently, the adhesive tape 8 and the cover 19 can smoothly expand and contract without being affected by the mounting substrate 13. Also, when the adhesive tape 8 and the cover 19 are expanded and contracted, the mounting substrate 13 is not affected by the displacement of the adhesive tape 8 and the cover 19. This ensures good reliability of the mounting substrate 13.

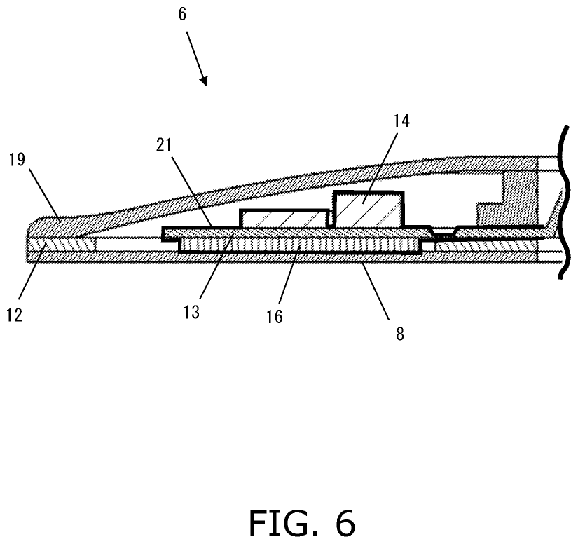

Furthermore, in this embodiment, as shown in FIG. 6, the mounting substrate 13 and the electronic components 14, the battery 15, and the spacers 16 mounted on the mounting substrate 13 are covered with a waterproof film 21. This minimizes degradation of the mounting substrate 13 due to moisture.

There is a risk that the waterproof films 21 covering the bottom side of the spacers 16 will be peeled off by friction when the adhesive tape 8 expands and contracts. However, the portion peeled off by friction will be on only the bottom side of the spacer 16, and no peeling will occur in the thickness direction or in the upper surface portion. This means that it is possible to prevent the function of the waterproof film 21 from being diminished. The spacers 16 are formed by using a flexible material such as an elastomer, rubber, or silicone.

### (2-2) Configuration of Inserter 7

The inserter 7 included in the biological information measurement system 1A in this embodiment is a device used for attaching the biological information measurement device 6 to the upper arm of the user, and as shown in FIGS. 2, 3, and 7, the inserter 7 is mounted on the upper surface of the biological information measurement device 6.

The inserter 7 comprises a substantially circular base body 22 and a circular container-shaped top body 23, in a state of being attached to the biological information measurement device 6.

The base body 22 covers the top side of the cover 19 of the biological information measurement device 6 and is supported by the upper end portion of the guide needle insertion cylinder 18 via the cover 19. The base body 22 has a guide cylinder 24 protruding toward the top body 23 from near the approximate center of the substantially circular upper surface.

The top body 23 is mounted on the base body 22 and is flexible. As shown in FIG. 3, the top body 23 has a substantially cylindrical guide needle holding portion 25 that can move up and down on the inner peripheral surface side of the guide cylinder 24.

The substantially cylindrical guide needle holding portion 25 holds the guide needle 26 along the center axis of the substantially cylindrical shape. When the central portion of the upper surface of the top body 23 is pressed toward the base body 22, the guide needle 26 transfers the sensor 17 to a side of the skin 5 of user. That is, the guide cylinder 24 provided to the base body 22 is provided in order to prevent the misalignment of the sensor 17 during transfer, and to perform positioning in the transfer of the guide needle holding portion 25.

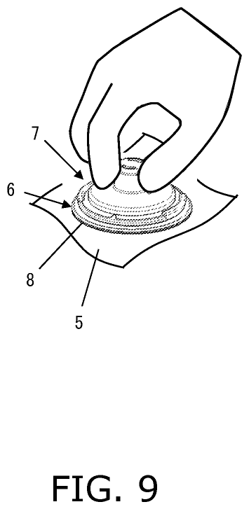

In the above configuration, the user first takes the biological information measurement device 6 to which the inserter 7 is mounted out of the container 4 shown in FIG. 1, and then makes use of the stickiness of the adhesive tape 8 to affix the biological information measurement device 6 to the skin 5 of the upper arm while the inserter 7 is still attached, as shown in FIG. 9.

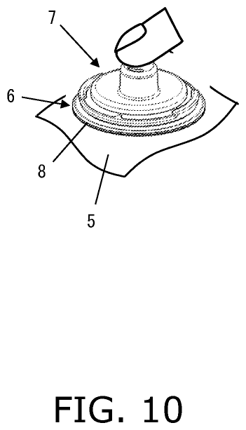

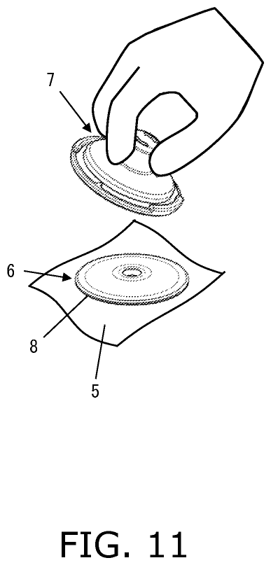

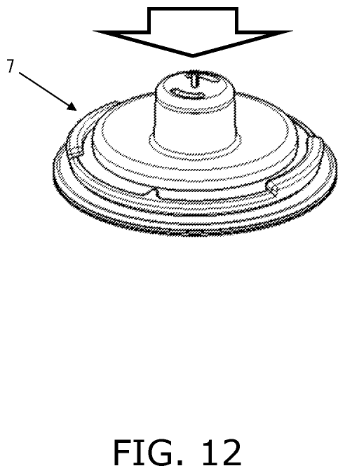

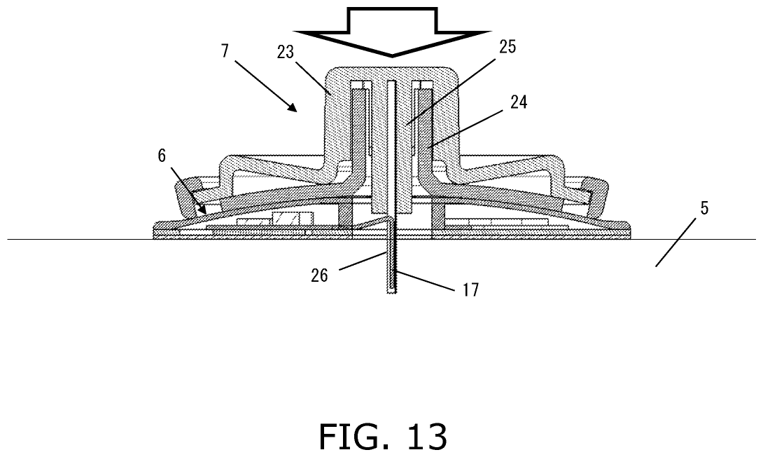

Next, as shown in FIGS. 10, 12, and 13, when the user pushes down the upper end surface of the inserter 7, the guide needle holding portion 25 of the inserter 7 slides through the guide cylinder 24 as shown in FIG. 13 and pushed down, that is, toward the skin 5. Consequently, the guide needle 26 pushes the sensor 17 toward the skin 5, which allows the distal end part of the sensor 17 to be transferred into the skin 5.

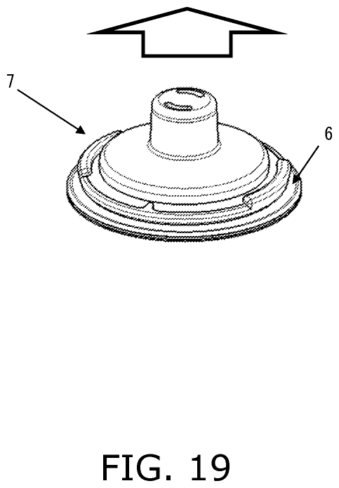

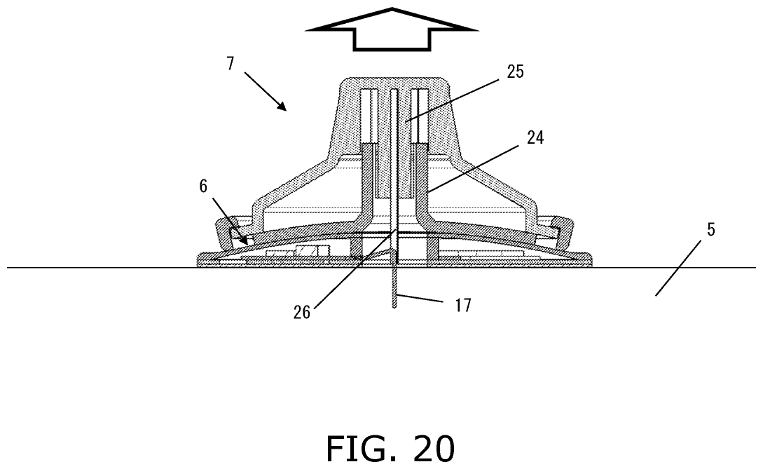

After this, the guide needle holding portion 25 of the inserter 7 automatically returns to its upper steady-state position under the elastic force (restoring force) of the inserter 7, as shown in FIGS. 19 and 20. This causes the distal end of the guide needle 26 that has been inserted into the skin 5 to be withdrawn from the skin 5.

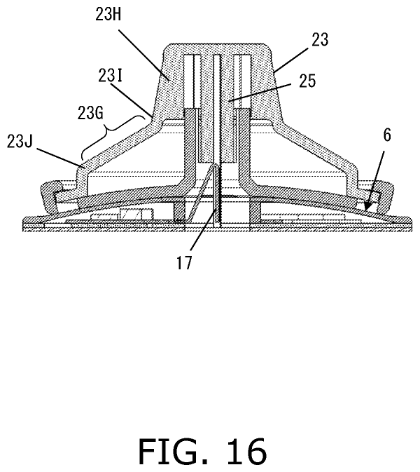

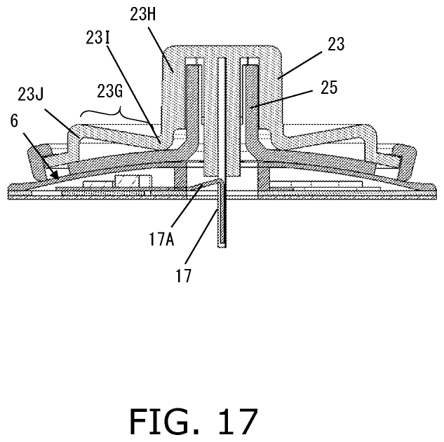

Also, as shown in FIGS. 16 and 17, in the top body 23, the wall thickness of a hinge portion 23G is less than the wall thickness of a pressing portion 23H, and the wall thickness of the hinge portion 23G is greater than the wall thickness of a bent portion 23I and a bent portion 23J.

Consequently, when the upper end surface of the top body 23 is pushed down, the thin hinge portion 23G is elastically deformed and pushed down, while the portion of the pressing portion 23H disposed on the outer peripheral side of the guide needle holding portion 25 hardly deforms at all due to its greater thickness.

Similarly, in the elastic deformation of the thin hinge portion 23G, the bent portions 23I and 23J, which are thicker than the hinge portion 23G, undergo almost no elastic deformation, and the pressing portion 23H is pushed down, with the bent portions 23I and 23J serving as fulcrums.

As a result, a stronger elastic force can be obtained for returning the top body 23 to its steady state, and the guide needle holding portion 25 can be operated more accurately and stably in the up and down direction.

Also, when the sensor 17 is inserted into the wound formed by puncturing the skin 5 with the guide needle 26 and the guide needle 26 is withdrawn from the skin 5 in the above operation, the sensor 17 is left behind under the skin 5.

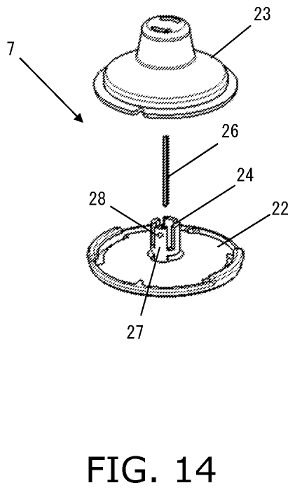

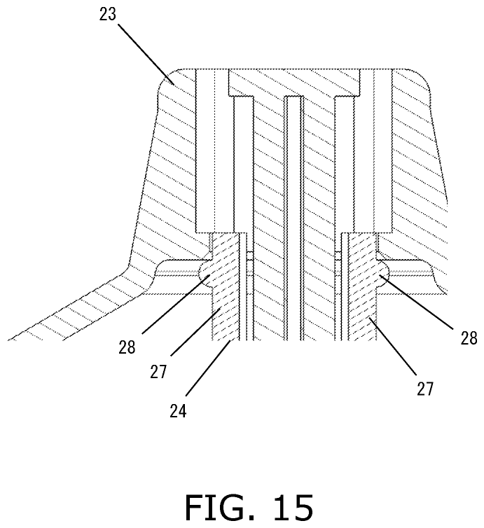

The inserter 7 in this embodiment has the configuration shown in FIGS. 14 and 15 in order for the guide needle holding portion 25 holding the guide needle 26 at the distal end to be pushed stably toward the skin 5.

Specifically, the guide cylinder 24 of the base body 22 has, on the upper end side thereof, a split piece 27 that is divided into four pieces in the circumferential direction. The split piece 27 has protrusions 28 that protrude in the outer peripheral direction (outward in the radial direction).

Therefore, when the top body 23 is pushed down, the protrusions 28 of the split piece 27 are pushed inward in the radial direction. Once the pressing force of this push operation overcomes the protrusions 28, it is quickly transmitted to the guide needle holding portion 25, so a constant pressing load can be ensured. Therefore, the guide needle 26 is reliably pushed in toward the skin 5.

In this embodiment, the configuration as shown in FIGS. 16 to 18 is employed so that when the sensor 17 is transferred under the skin 5 by pushing down the guide needle 26 in this way, the sensor 17 will not be broken by a sudden push on the guide needle 26.

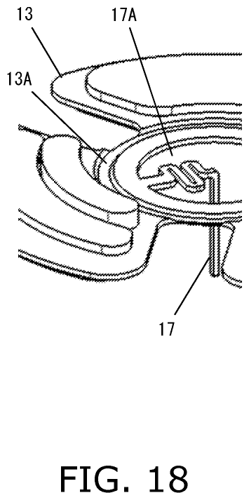

That is, as shown in FIG. 18, a meandering elastic portion 17A is provided near the portion of the sensor 17 that comes into contact with the distal end of the guide needle holding portion 25. Consequently, even if the distal end of the sensor 17 is rapidly pushed into the skin 5, it is possible to prevent a part of the sensor 17 from being damaged and the wire disconnected.

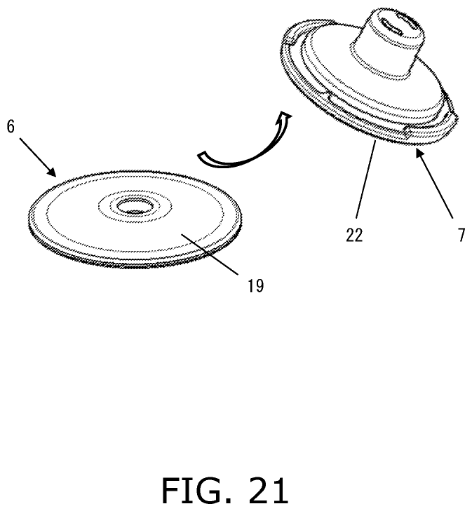

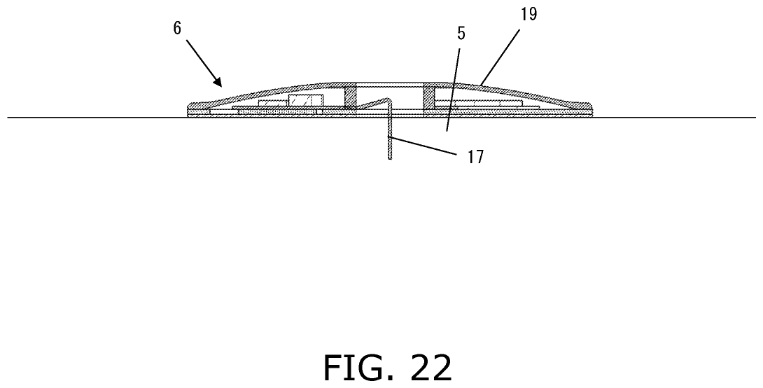

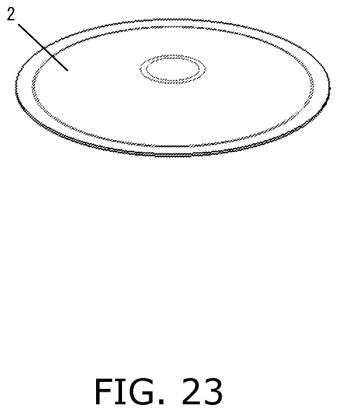

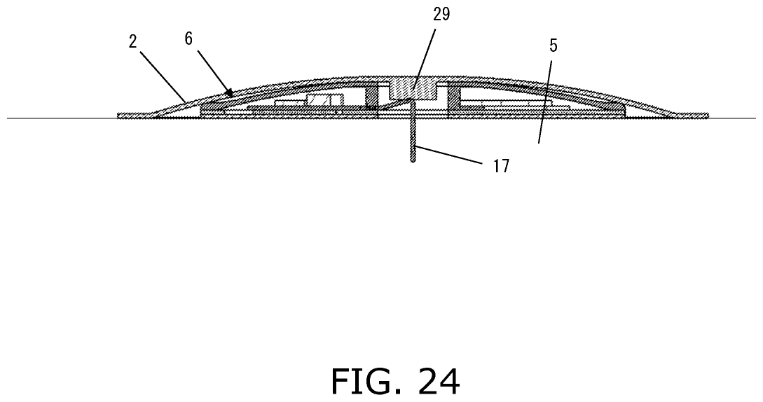

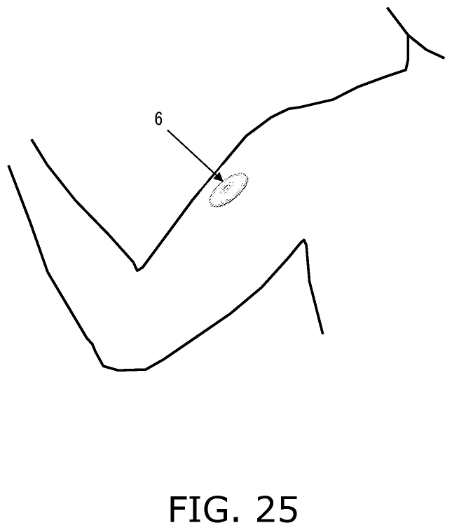

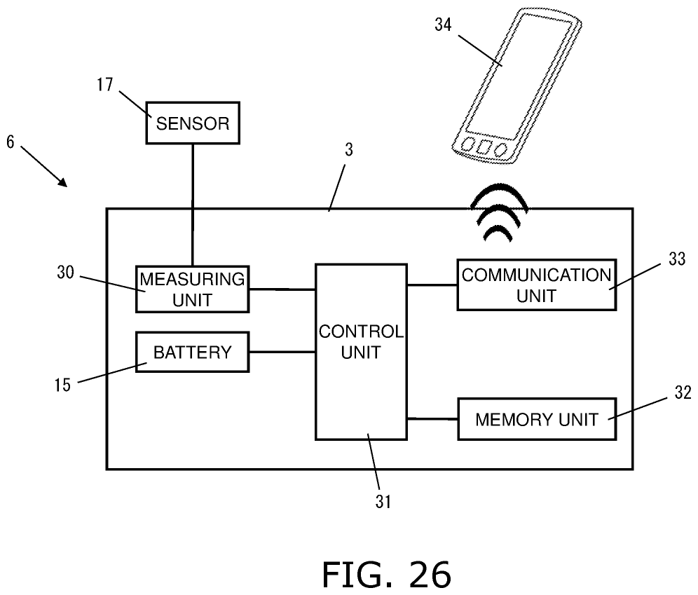

After this, as shown in FIG. 20, the distal end of the sensor 17 is transferred under the skin 5 and the top body 23 returns to its steady-state position under elastic force, after which the inserter 7 is removed from the upper surface (cover 19) of the biological information measurement device 6 as shown in FIGS. 21 and 22. That is, part of the base body 22 of the inserter 7 is bonded to the cover 19 in a state of point contact. This allows the inserter 7 to be easily peeled off from the biological information measurement device 6, as shown in FIG. 21.

At this point, the adhesive strength of the point contact portion between the cover 19 and the base body 22 of the inserter 7 is sufficiently less than the adhesive strength between the cover 19 and the guide needle insertion cylinder 18, the adhesive strength between the guide needle insertion cylinder 18 and the mounting substrate 13, the adhesive strength between the mounting substrate 13 and the base 12, the adhesive strength between the base 12 and the adhesive tape 8, and the adhesive strength between the adhesive tape 8 and the skin 5 shown in FIG. 5.

Consequently, as shown in FIG. 21, the inserter 7 can be easily peeled off from the biological information measurement device 6.

When the inserter 7 is removed from the biological information measurement device 6, the overtape 2 is attached to the top side of the cover 19 as shown in FIGS. 23 and 24.

The overtape 2 is formed from a stretchable material such as rubber or an elastomer. As shown in FIG. 24, a protrusion 29 that is inserted into the guide needle insertion cylinder 18 is provided to the portion of the overtape 2 corresponding to the guide needle insertion cylinder 18.

That is, the protrusion 29 comes into contact from above with the intermediate portion of the sensor 17 that the distal end of the guide needle holding portion 25 was in contact with when the biological information measurement device 6 was attached to the skin 5. This prevents the sensor 17 from inadvertently coming out upward.

The result of the above operation is that the biological information measurement device 6 is attached to the skin 5 of the upper arm of the user as shown in FIG. 25.

With the biological information measurement system 1A in this embodiment, the biological information measurement device 6 can be easily attached by hand to the skin 5 of the upper arm of the user by making use of the stickiness of the adhesive tape 8.

Also, in a state in which the biological information measurement device 6 to which the inserter 7 is attached has been affixed to the skin 5, the top body 23 is pushed in toward the skin 5 with the same hand used to affix the inserter 7, thereby allowing the sensor 17 to be easily transferred under the skin 5 by the guide needle 26.

Furthermore, after this, the inserter 7 bonded to the cover 19 of the biological information measurement device 6 by point contact can be easily removed by hand, and as shown in FIG. 24, the attachment of the biological information measurement device 6 to the skin 5 can be completed by affixing the overtape 2 on the upper surface of the biological information measurement device 6 by hand.

As shown in FIG. 24, after being attached, the biological information measurement device 6 has a shape that is thin and gradually thickens toward the central portion of the approximate disk shape. As a result, it is possible to avoid a shape in which the attached biological information measurement device 6 protrudes from the surface of the skin 5, so that the device is not unattractive when worn.

### Main Features

The adhesive tape 8, the cover 19, and the base 12 of the biological information measurement device 6 in this embodiment are formed from members that are stretchable in the direction running along the surface of the skin 5 and in the direction intersecting the surface of the skin 5 when the device is being worn, and are joined around the outer peripheral part. The mounting substrate 13, which does not stretch in the direction running along the surface of the skin 5, is fixed to the central portion of the adhesive tape 8, the cover 19, and the base 12 via the guide needle insertion cylinder 18.

Specifically, when the biological information measurement device 6 is attached to the skin 5, the adhesive tape 8, the cover 19, and the overtape 2 of the biological information measurement device 6 are pliant and stretchable.

Consequently, the biological information measurement device 6 can be attached in a state of conforming to the skin 5 to which the device is attached, which affords a biological information measurement device 6 that is more comfortable to wear.

Also, since the outer peripheral edge portions can be stretched out and attached to the skin 5 with respect to the central portion of the adhesive tape 8, the cover 19, and the overtape 2, there is stretchability in the direction running along the surface of the skin 5 and flexibility in the direction intersecting the surface of the skin 5, so the fit feels better when the device is attached.

Furthermore, in this embodiment, since the adhesive tape 8, the cover 19, and the overtape 2 can expand and contract according to the movements of the wearer's body during actual use, the user is less likely to feel any discomfort during body movements, and this further enhances wearing comfort.

In order to obtain this effect, as shown in FIG. 4, it is preferable for the taller of the battery 15 and the electronic components 14 to be mounted on the mounting substrate 13 at a position closer to the guide needle insertion cylinder 18.

As described above, the adhesive tape 8, the cover 19, and the base 12 in this embodiment are formed from a material, such as an elastomer, that is stretchable in the direction running along the surface of the skin 5, and in the direction intersecting the skin 5.

On the other hand, as described above, the mounting substrate 13 is formed from a polyimide or polyester resin used for a flexible substrate. These materials are flexible in the bending direction, but are not stretchable in a direction substantially parallel to the substrate surface.

The adhesive tape 8, the cover 19, the base 12, and the mounting substrate 13 are flexible in the bending direction. The mounting substrate 13 is placed on the adhesive tape 8 in an unbonded state via the spacers 16.

Consequently, the device can be affixed so that it fits the curves of the skin 5 where the device is attached, without being affected by the mounting substrate 13.

In this embodiment, as shown in FIG. 5, the mounting substrate 13 has a thin portion 13A provided around the periphery of the guide needle insertion cylinder 18, and near the guide needle insertion cylinder 18 (see FIG. 18). This allows the mounting substrate 13 to be bent more flexibly in the bending direction intersecting the surface of the substrate.

Also, the adhesive tape 8, the cover 19, and the base 12 are stretchable in a direction parallel to the surface of the skin 5 when the device is attached. This allows them to be affixed to the bonding site while being stretched out to both sides like a bandage tape.

At this point, the mounting substrate 13, which is not stretchable in the direction parallel to the surface of the skin 5 in the attached state, is fixed only in the central parts of the adhesive tape 8, the cover 19 and the base 12, which are stretchable in the direction parallel to the surface of the skin 5 in the attached state.

Consequently, the mounting substrate 13 will not be stretched out in the horizontal direction even when it is stretched out to both sides and attached to the skin 5.

Also, bending in the up and down direction is possible, matching the curved shape of the surface of the skin 5 to which the device is attached, the biological information measurement device 6 can be attached in a state of being fitted to the surface of the skin 5, similar to how a bandage tape is attached.

### INDUSTRIAL APPLICABILITY

The present invention is expected to find use as a biological information measurement device for continuous blood glucose measurement, for example.

### REFERENCE SIGNS LIST

1A biological information measurement system

1 sterilized packaging

2 overtape

3 sterilized packaging

4 container

5 skin

6 biological information measurement device

7 inserter

8 adhesive tape

8a through-hole

9 outer peripheral circle portion

10 inner peripheral circle portion

11 bridge portions

12 base

13 mounting substrate

13A thin portion

14 electronic component

15 battery

16 spacer

17 sensor

17A elastic portion

18 guide needle insertion cylinder (example of substrate holding portion)

19 cover

20 through-hole (through-hole)

21 waterproof film

22 base body

23 top body

23G hinge portion

23H pressing portion

23I bent portion

23J bent portion

24 guide cylinder

25 guide needle holding portion

26 guide needle

27 split piece

28 protrusion

29 protrusion

30 measuring unit

31 control unit

32 memory unit

33 communication unit

34 mobile device

A, B, C, D, E, F joints

1. A biological information measurement device, comprising:
an adhesive tape that has on its bottom side an adhesive surface to be affixed to a skin of a user;
a mounting substrate that is disposed on a top side of the adhesive tape;
a substrate holding portion that is provided on the top side of the adhesive tape and that holds the mounting substrate;
a sensor electrically that is connected to the mounting substrate and is used to measure biological information; and
a cover that covers a top side of the mounting substrate,
wherein a bottom side of the mounting substrate is in a state of not being affixed to the top side of the adhesive tape, and
the adhesive tape and the cover are more stretchable than the mounting substrate.

2. The biological information measurement device according to claim 1,
wherein the mounting substrate has low stretchability in a direction substantially parallel to a surface of the skin in a state in which the adhesive tape has been applied to the skin.

3. The biological information measurement device according to claim 1,
further comprising spacers disposed between a lower surface of the mounting substrate and an upper surface of the adhesive tape.

4. The biological information measurement device according to claim 3,
wherein the spacers are bonded to the lower surface of the mounting substrate on the top side, and to the upper surface of the adhesive tape on the bottom side.

5. The biological information measurement device according to claim 3,
wherein the mounting substrate has a plurality of substantially fan-shaped parts, and the spacers are substantially fan-shaped to match a shape of the mounting substrate.

6. The biological information measurement device according to claim 1,
further comprising a base that is disposed between the adhesive tape and the mounting substrate, and that is substantially annular in shape.

7. The biological information measurement device according to claim 6,
wherein the adhesive tape and the cover are bonded together other near an outer peripheral edge portion, with a substantially annular base interposed therebetween.

8. The biological information measurement device according to claim 6,
wherein the base has a substantially annular outer peripheral circle portion, an inner peripheral circle portion that is disposed concentrically in a radial direction with the outer peripheral circle portion, and a plurality of bridges that connect the outer peripheral circle portion and the inner peripheral circle portion at substantially equal angular intervals.

9. The biological information measurement device according to claim 1,
wherein the substrate holding portion has a substantially cylindrical shape, and
the lower surface of the cover is bonded to the substantially cylindrical upper end surface of the substrate holding portion, near a central portion of the cover.

10. The biological information measurement device according to claim 1, further comprising:
an overtape that is bonded to an upper surface of the cover; and
a protrusion that is provided to a portion of the overtape corresponding to the substrate holding portion, and is inserted into the substrate holding portion.

11. The biological information measurement device according to claim 1, further comprising:
electronic components mounted on the top side of the mounting substrate; and
a waterproof film that covers the mounting substrate and the electronic components.

12. A biological information measurement system comprising a biological information measurement device that measures biological information about a user, and an inserter that is placed on an upper surface of the biological information measurement device and mounts the biological information measurement device on a skin of a user,
wherein the biological information measurement device has:
an adhesive tape that has on its bottom side an adhesive surface to be affixed to the skin of a user;
a mounting substrate that is disposed on a top side of the adhesive tape;
a substrate holding portion that is provided on the top side of the adhesive tape and that holds the mounting substrate;
a sensor electrically that is connected to the mounting substrate and is used to measure biological information; and
a cover that covers a top side of the mounting substrate and has a through-hole formed in the substrate holding portion,
wherein the inserter has:
a base body that is mounted on an upper surface of the cover of the biological information measurement device at a position including the through-hole, and that is supported by the upper end portion of the substrate holding portion of the biological information measurement device; and
a flexible top body that is mounted on the base body and is elastically deformed when pressed during a mounting of the biological information measurement device on the skin.

13. The biological information measurement system according to claim 12,
wherein the base body has a guide cylinder protruding toward the top body side,
the top body has a guide needle holding portion configured to move in the puncture direction along the inside of the guide cylinder, and a guide needle that is held by the guide needle holding portion and is configured to puncture the skin, and
when the top body is pressed toward a side of the base body during the mounting of the biological information measurement device on the skin, the sensor of the biological information measurement device is transferred to the skin side along with the guide needle.

14. The biological information measurement system according to claim 12,
wherein the base body is detachably attached to the upper surface of the cover.

15. The biological information measurement system according to claim 14,
wherein the base body is bonded to the upper surface of the cover in a state of point contact.

16. The biological information measurement system according to claim 14,
wherein an adhesive strength of the base body to the upper surface of the cover is less than an adhesive strength of the adhesive tape.

17. An inserter used to attach a biological information measurement device to a human body, comprising:
a base body that is mounted on an upper surface of the biological information measurement device; and
a flexible top body that is mounted on the base body and is elastically deformed during a mounting of the biological information measurement device on a skin,
wherein the base body has a guide cylinder protruding toward a side of the top body,
the top body has a guide needle holding portion configured to move in a puncture direction along an inside of the guide cylinder, and a guide needle that is held by the guide needle holding portion and is configured to puncture the skin, and
when the top body is pressed toward a side of the base body during the mounting of the biological information measurement device on the skin, the sensor of the biological information measurement device is transferred to a side of the skin along with the guide needle.

18. The inserter according to claim 17,
wherein the base body is detachably attached to an upper surface of the biological information measurement device.

19. The inserter according to claim 18,
wherein the base body is bonded to an upper surface of the cover in a state of point contact.

20. The inserter according to claim 18,
wherein an adhesive strength of the base body to the upper surface of the cover is less than an adhesive strength of the adhesive tape.
